(19) United States
(12) Patent Application Publication
(10) Pub. No.: US 2009/0025107 A1
(43) Pub. Date: Jan. 22, 2009

(54) Polypeptides Having Organophosphorus Hydrolase Activity and Polynucleotides Encoding Same

(75) Inventors: Lars Henrik Oestergaard, Charlottenlund (DK); Soeren Flensted Lassen, Farum (DK); Steffen Danielsen, Copenhagen (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 12/278,354

(22) PCT Filed: Feb. 6, 2007
(86) PCT No.: PCT/DK07/00061

§ 371 Date: Aug. 5, 2008

(30) Foreign Application Priority Data
Feb. 6, 2006 (DK) PA 2006 00159

(51) Int. Cl. A01H 5/00; C12N 9/16; C07H 21/00; C12N 15/64; C12N 1/21; C12N 5/04

(52) U.S. Cl. 800/295; 435/196; 536/23.2; 435/252.3; 536/23.1; 435/410; 435/320.1

(57) ABSTRACT

The present invention relates to isolated polypeptides having organophosphorus hydrolase (OPH) activity and isolated polynucleotides encoding the polypeptides. The invention also relates to nucleic acid constructs, vectors, and host cells comprising the polynucleotides as well as methods for producing and using the polypeptides.

### SEQUENCE LISTING

This application contains a Sequence Listing in computer readable form. The computer readable form is incorporated herein by reference.

### DEPOSIT OF BIOLOGICAL MATERIAL

The following biological material has been deposited under the terms of the Budapest Treaty with the DSMZ-Deutsche Sammiung von Mikroorganismen und Zellkulturen GmbH, Mascheroder Weg 1b, D-38124 Braunschweig and given the following accession number:

| Deposit | Accession Number | Date of Deposit |
|---|---|---|
| Escherichia coli | DSM 17765 | 5 Dec. 2005 |

The strain has been deposited under conditions that assure that access to the culture will be available during the pendency of this patent application to one determined by the Commissioner of Patents and Trademarks to be entitled thereto under 37 C.F.R. §1.14 and 35 U.S.C. §122. The deposit represents a substantially pure culture of the deposited strain. The deposit is available as required by foreign patent laws in countries wherein counterparts of the subject application or its progeny are filed. However, it should be understood that the availability of a deposit does not constitute a license to practice the subject invention in derogation of patent rights granted by governmental action.

### FIELD OF THE INVENTION

The present invention relates to isolated polypeptides having organophosphorus hydrolase activity and isolated polynucleotides encoding the polypeptides. The invention also relates to nucleic acid constructs, vectors, and host cells comprising the polynucleotides as well as methods for producing and using the polypeptides.

### BACKGROUND OF THE INVENTION

Organophosphates belong to a group of chemicals which includes nerve agents, insecticidal and fungicidal agents. These agents are highly toxic and thus means for detoxifying such agents are highly desirable. Poisoning with organophosphates presents a problem in agriculture as well as for military personnel exposed to organophosphates used in chemical warfare. One way of detoxifying such agents involves enzymatic degradation of organophosphates. Enzymes capable of hydrolyzing organophosphates detoxify a variety of nerve agents as well as commonly used pesticides. Organophosphorus hydrolase (OPH) has been widely studied for its activity on organophosphorus (OP) pesticides and its ability to hydrolyse nerve agents. Suitable enzymes for degrading organophosphate residues include OP hydrolases from bacteria (Harper et al., 1988, Applied and Environmental Microbiology 54: 2586-2589; Mulbury and Karns, 1989, J. Bacteriol. 171: 6740-6746; Mulbury, 1992, Gene 121: 149-153; Mulbury and Kearney, 1991, Crop Protection 10: 334-345; Cheng et al., 1999, Chemico-Biological Interactions 119-120:455-462; Horne et al., 2002, Applied and Enveronmental Microbiology 68: 3371-3376; U.S. Pat. No. 5,484,728; U.S. Pat. No. 5,589,386), vertebrates (Wang et al. 1998, J. Biochem. and Mol. Toxicology 12: 213-217) and OP resistant insects (WO 95/19440 and WO97/19176). It is desirable to find alternative and/or improved OP hydrolyzing enzymes that can conveniently be produced in sufficient amounts.

It is an object of the present invention to provide polypeptides having organophosphatase activity and polynucleotides encoding the polypeptides. Also such organophosphatase enzymes should readily be expressed and purified from a fungal expression system.

### SUMMARY OF THE INVENTION

The present invention relates in a first aspect to isolated polypeptide having organophosphatase activity, selected from the group consisting of:

(a) a polypeptide comprising an amino acid sequence which has at least 87% identity with amino acids 36 to 334 of SEQ ID NO: 2;

(b) a polypeptide which is encoded by a polynucleotide which hybridizes under at least medium stringency conditions with (i) nucleotides 106 to 1002 of SEQ ID NO: 1, or (ii) a complementary strand of (i);

(c) a variant comprising a conservative substitution, deletion, and/or insertion of one or more amino acids of amino acids 36 to 334 of SEQ ID NO: 2;

In a second aspect the present invention relates to an isolated polynucleotide encoding an organophosphatase, selected from the group consisting of:

(a) a nucleic acid sequence encoding a polypeptide comprising an amino acid sequence which has at least 87% identity with amino acids 36 to 334 of SEQ ID NO: 2;

(b) a nucleic acid sequence having at least 81% identity with the nucleic acid sequence 106 to 1002 of SEQ ID NO: 1;

(c) a nucleic acid sequence which hybridizes under medium stringency conditions with (i) the nucleic acid sequence from position 106 to 1002 of SEQ ID NO: 1, (ii) a subsequence of (i) of at least 100 nucleotides, or (iii) a complementary strand of (i) or (ii);

(d) an allelic variant of (a), (b), (c);

(e) a subsequence of (a), (b), (c), or (d), wherein the subsequence encodes a polypeptide fragment which has organophosphatase activity.

The present invention also relates to nucleic acid constructs, recombinant expression vectors, and recombinant host cells comprising polynucleotides encoding the polypeptides of the invention.

The present invention also relates to methods for producing such polypeptides having OPH activity comprising (a) cultivating a host cell comprising a nucleic acid construct comprising a polynucleotide encoding the polypeptide under conditions conducive for production of the polypeptide; and (b) recovering the polypeptide.

In a further aspect the present invention relates to a method for producing a polynucleotide having a mutant nucleotide sequence, comprising (a) introducing at least one mutation into the mature polypeptide coding sequence of SEQ ID NO: 1, wherein the mutant nucleotide sequence encodes a polypeptide comprising amino acids 36 to 334 of SEQ ID NO: 2; and (b) recovering the polynucleotide comprising the mutant nucleotide sequence.

In a still further aspect the present invention relates to a method for producing the polypeptide of the invetnion, comprising (a) cultivating a transgenic plant or a plant cell comprising a polynucleotide encoding a polypeptide having organophosphatase activity of the present invention under conditions conducive for production of the polypeptide; and (b) recovering the polypeptide.

In an even further aspect the present invention relates to a method for producing the polypeptide of the invention, comprising (a) cultivating an algae comprising a polynucleotide encoding a polypeptide having organophosphatase activity of the present invention under conditions conducive for production of the polypeptide; and (b) recovering the polypeptide.

The present invention also relates to a use of the polypeptide according to the invention for hydrolysing an organophosphorus molecule.

In a still further aspect the invention relates to a composition for hydrolysing an organophosphatase molecule, said composition comprising a polypeptide according to the invention and one or more acceptable carriers.

In another aspect the invention relates to a transgenic plant which produces a polypeptide according to the invention.

Another aspect relates to a method for hydrolysing an organophosphatase molecule, said method comprising exposing the organophosphatase molecule to the polypeptide according to the invention or the composition according to the invention or the transgenic plant according to the invention.

### DEFINITIONS

Organophosphatase activity: The term “organophosphatase activity” is defined herein as an organophosphorus hydrolase (OPH) (EC 3.1.8.1) activity which catalyzes the hydrolysis of organophosphorus compounds. These enzymes are also known as aryldialkylphosphatases. OPH's act on organophosphorus compounds, such as paraoxin, including esters of phosphonic and phosphinic acids. For purposes of the present invention, organophosphatse activity is determined according to the procedure described in EnzChek®Paraoxonase assay kit available from Molecular Probes™. An alternative assay for determining OPH activity is described in Cho et al., 2004, Applied and Environmental Microbiology, vol. 70(8):4681-4685.

The polypeptides of the present invention have at least 20%, preferably at least 40%, more preferably at least 50%, more preferably at least 60%, more preferably at least 70%, more preferably at least 80%, even more preferably at least 90%, most preferably at least 95%, and even most preferably at least 100% of the organophosphatase activity of the polypeptide comprising the amino acid sequence shown as amino acids 36 to 334 of SEQ ID NO: 2.

Isolated polypeptide: The term “isolated polypeptide” as used herein refers to a polypeptide which is at least 20% pure, preferably at least 40% pure, more preferably at least 60% pure, even more preferably at least 80% pure, most preferably at least 90% pure, and even most preferably at least 95% pure, as determined by SDS-PAGE.

Substantially pure polypeptide: The term “substantially pure polypeptide” denotes herein a polypeptide preparation which contains at most 10%, preferably at most 8%, more preferably at most 6%, more preferably at most 5%, more preferably at most 4%, at most 3%, even more preferably at most 2%, most preferably at most 1%, and even most preferably at most 0.5% by weight of other polypeptide material with which it is natively associated. It is, therefore, preferred that the substantially pure polypeptide is at least 92% pure, preferably at least 94% pure, more preferably at least 95% pure, more preferably at least 96% pure, more preferably at least 96% pure, more preferably at least 97% pure, more preferably at least 98% pure, even more preferably at least 99%, most preferably at least 99.5% pure, and even most preferably 100% pure by weight of the total polypeptide material present in the preparation.

The polypeptides of the present invention are preferably in a substantially pure form. In particular, it is preferred that the polypeptides are in “essentially pure form”, ie., that the polypeptide preparation is essentially free of other polypeptide material with which it is natively associated. This can be accomplished, for example, by preparing the polypeptide by means of well-known recombinant methods or by classical purification methods.

Herein, the term “substantially pure polypeptide” is synonymous with the terms “isolated polypeptide” and “polypeptide in isolated form.”

Identity: The relatedness between two amino acid sequences is described by the parameter “identity”.

For purposes of the present invention, the alignment of two amino acid sequences is determined by using the Needle program from the EMBOSS package (http://emboss.org) version 2.8.0. The Needle program implements the global alignment algorithm described in Needleman, S. B. and Wunsch, C. D. (1970) J. Mol. Biol. 48, 443-453. The substitution matrix used is BLOSUM62, gap opening penalty is 10, and gap extension penalty is 0.5.

The degree of identity between an amino acid sequence of the present invention (“invention sequence”); e.g. amino acids 36 to 334 of SEQ ID NO: 2 and a different amino acid sequence (“foreign sequence”) is calculated as the number of exact matches in an alignment of the two sequences, divided by the length of the “invention sequence” or the length of the “foreign sequence”, whichever is the shortest. The result is expressed in percent identity.

An exact match occurs when the “invention sequence” and the “foreign sequence” have identical amino acid residues in the same positions of the overlap (in the alignment example below this is represented by “|”). The length of a sequence is the number of amino acid residues in the sequence.

In the purely hypothetical alignment example below, the overlap is the amino acid sequence “HTWGER-NL” of Sequence 1; or the amino acid sequence “HGWGEDANL” of Sequence 2. In the example a gap is indicated by a “-”.

### Hypothetical Alignment Example:

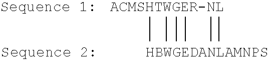

In a particular embodiment, the percentage of identity of an amino acid sequence of a polypeptide with, or to, amino acids 36 to 334 of SEQ ID NO: 2 is determined by i) aligning the two amino acid sequences using the Needle program, with the BLOSUM62 substitution matrix, a gap opening penalty of 10, and a gap extension penalty of 0.5; ii) counting the number of exact matches in the alignment; iii) dividing the number of exact matches by the length of the shortest of the two amino acid sequences, and iv) converting the result of the division of iii) into percentage. The percentage of identity to, or with, other sequences of the invention such as amino acids 1-334 of SEQ ID NO: 2 are calculated in an analogous way.

For purposes of the present invention, the degree of identity between two nucleotide sequences is determined by the Wilbur-Lipman method (Wilbur and Lipman, 1983, Proceedings of the National Academy of Science USA 80: 726-730) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters are Ktuple=3, gap penalty=3, and windows=20.

Polypeptide Fragment: The term “polypeptide fragment” is defined herein as a polypeptide having one or more amino acids deleted from the amino and/or carboxyl terminus of SEQ ID NO: 2 or a homologous sequence thereof, wherein the fragment has OPH activity.

Subsequence: The term “subsequence” is defined herein as a nucleotide sequence having one or more nucleotides deleted from the 5′ and/or 3′ end of SEQ ID NO: 1 or a homologous sequence thereof, wherein the subsequence encodes a polypeptide fragment having OPH activity.

Allelic variant: The term “allelic variant” denotes herein any of two or more alternative forms of a gene occupying the same chromosomal locus. Alletic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Substantially pure polynucleotide: The term “substantially pure polynucleotide” as used herein refers to a polynucleotide preparation free of other extraneous or unwanted nucleotides and in a form suitable for use within genetically engineered protein production systems. Thus, a substantially pure polynucleotide contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polynucleotide material with which it is natively associated. A substantially pure polynucleotide may, however, include naturally occurring 5′ and 3′ untranslated regions, such as promoters and terminators. It is preferred that the substantially pure polynucleotide is at least 90% pure, preferably at least 92% pure, more preferably at least 94% pure, more preferably at least 95% pure, more preferably at least 96% pure, more preferably at least 97% pure, even more preferably at least 98% pure, most preferably at least 99%, and even most preferably at least 99.5% pure by weight. The polynucleotides of the present invention are preferably in a substantially pure form. In particular, it is preferred that the polynucleotides disclosed herein are in “essentially pure form”, i.e., that the polynucleotide preparation is essentially free of other polynucleotide material with which it is natively associated. Herein, the term “substantially pure polynucleotide” is synonymous with the terms “isolated polynucleotide” and “polynucleotide in isolated form.” The polynucleotides may be of genomic, cDNA, RNA, semisynthetic, synthetic origin, or any combinations thereof.

Nucleic acid construct: The term “nucleic acid construct” as used herein refers to a nucleic acid molecule, either single- or double-stranded, which is isolated from a naturally occurring gene or which is modified to contain segments of nucleic acids in a manner that would not otherwise exist in nature. The term nucleic acid construct is synonymous with the term “expression cassette” when the nucleic acid construct contains the control sequences required for expression of a coding sequence of the present invention.

Control sequence: The term “control sequences” is defined herein to include all components, which are necessary or advantageous for the expression of a polynucleotide encoding a polypeptide of the present invention. Each control sequence may be native or foreign to the nucleotide sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a polypeptide.

Operably linked: The term “operably linked” denotes herein a configuration in which a control sequence is placed at an appropriate position relative to the coding sequence of the polynucleotide sequence such that the control sequence directs the expression of the coding sequence of a polypeptide.

Coding sequence: When used herein the term “coding sequence” means a nucleotide sequence, which directly specifies the amino acid sequence of its protein product. The boundaries of the coding sequence are generally determined by an open reading frame, which usually begins with the ATG start codon or alternative start codons such as GTG and TTG. The coding sequence may a DNA, cDNA, or recombinant nucleotide sequence.

Expression: The term “expression” includes any step involved in the production of the polypeptide including, but not limited to, transcription, post-transcriptional modification, translation, post-translational modification, and secretion.

Expression vector: The term “expression vector” is defined herein as a linear or circular DNA molecule that comprises a polynucleotide encoding a polypeptide of the invention, and which is operably linked to additional nucleotides that provide for its expression.

Host cell: The term “host cell”, as used herein, includes any cell type which is susceptible to transformation, transfection, transduction, and the like with a nucleic acid construct comprising a polynucleotide of the present invention.

Modification: The term “modification” means herein any chemical modification of the polypeptide consisting of the amino acids 1 to 334 of SEQ ID NO: 2 as well as genetic manipulation of the DNA encoding that polypeptide. The modification(s) can be substitution(s), deletion(s) and/or insertions(s) of the amino acid(s) as well as replacement(s) of amino acid side chain(s).

Artificial variant: When used herein, the term “artificial variant” means a polypeptide having OPH activity produced by an organism expressing a modified nucleotide sequence of SEQ ID NO: 1. The modified nucleotide sequence is obtained through human intervention by modification of the nucleotide sequence disclosed in SEQ ID NO: 1, more preferably in position 103-1006 of SEQ ID NO: 1.

### DETAILED DESCRIPTION OF THE INVENTION

Polypeptides having OPH Activity

The present invention relates to isolated polypeptides having organophosphatase activity comprising an amino acid sequence which has a degree of identity to amino acids 36 to 334 of SEQ ID NO: 2 (i.e., the mature polypeptide) of at least 87%, preferably at least 88%, more preferably at least 89%, more preferably at least 90%, more preferably at least 91%, more preferably at least 92%, more preferably at least 93%, more preferably at least 95%, even more preferably at least 97%, and even most preferably at least 99% (hereinafter “homologous polypeptides”). In a preferred aspect, the homologous polypeptides have an amino acid sequence which differs by ten amino acids, preferably by five amino acids, more preferably by four amino acids, even more preferably by three amino acids, most preferably by two amino acids, and even most preferably by one amino acid from amino acids 36 to 334 of SEQ ID NO: 2.

A polypeptide of the present invention preferably comprises the amino acid sequence from position 36 to 334 of SEQ ID NO: 2 or an allelic variant thereof; or a fragment thereof that has OPH activity. The data presented herein have identified the N-terminal of the mature polypeptide to be either ATQQRTQ or TQQRTQV, which shows that the mature polypeptide having OPH activity most likely starts at position 35 in SEQ ID NO: 2, however the start could also be at position 36 since a fragment having an N-terminal starting at position 36 in SEQ ID NO: 2 was also observed. In a one aspect, the polypeptide of the invention therefore consists of the amino acid sequence of SEQ ID NO: 2. In another aspect, the polypeptide comprises amino acids 36 to 334 of SEQ ID NO: 2, or an allelic variant thereof; or a fragment thereof that has OPH activity. In another preferred aspect, the polypeptide comprises amino acids 35 to 334 of SEQ ID NO: 2 or an allelic variant thereof; or a fragment thereof that has OPH activity.

In a further aspect, the present invention relates to isolated polypeptides having OPH activity which are encoded by polynucleotides which hybridize under very low stringency conditions, preferably low stringency conditions, more preferably medium stringency conditions, more preferably medium-high stringency conditions, even more preferably high stringency conditions, and most preferably very high stringency conditions with (i) nucleotides 106 to 1002 of SEQ ID NO: 1, (ii) a subsequence of (i) of at least 100 nucleotides or (iii) a complementary strand of (i) or (ii) (J. Sambrook, E. F. Fritsch, and T. Maniatis, 1989, Molecular Cloning, A Laboratory Manual, 2d edition, Cold Spring Harbor, N.Y.). A subsequence of SEQ ID NO: 1 contains at least 100 contiguous nucleotides or preferably at least 200 contiguous nucleotides. Moreover, the subsequence may encode a polypeptide fragment which has OPH activity.

For long probes of at least 100 nucleotides in length, very low to very high stringency conditions are defined as prehybridization and hybridization at 42° C. in 5×SSPE, 0.3% SDS, 200 μg/ml sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally.

For long probes of at least 100 nucleotides in length, the carrier material is finally washed three times each for 15 minutes using 2×SSC, 0.2% SDS preferably at least at 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency).

In a particular embodiment, the wash is conducted using 0.2×SSC, 0.2% SDS preferably at least at 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency). In another particular embodiment, the wash is conducted using 0.1×SSC, 0.2% SDS preferably at least at 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency).

The nucleotide sequence of SEQ ID NO: 1 or a subsequence thereof, as well as the amino acid sequence of SEQ ID NO: 2 or a fragment thereof, may be used to design a nucleic acid probe to identify and clone DNA encoding polypeptides having OPH activity from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 14, preferably at least 25, more preferably at least 35, and most preferably at least 70 nucleotides in length. It is, however, preferred that the nucleic acid probe is at least 100 nucleotides in length. For example, the nucleic acid probe may be at least 200 nucleotides, preferably at least 300 nucleotides, more preferably at least 400 nucleotides, or most preferably at least 500 nucleotides in length. Even longer probes may be used, e.g., nucleic acid probes which are at least 600 nucleotides, at least preferably at least 700 nucleotides, more preferably at least 800 nucleotides, or most preferably at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other organisms may, therefore, be screened for DNA which hybridizes with the probes described above and which encodes a polypeptide having OPH activity. Genomic or other DNA from such other organisms may be separated by agarose or polyacrylamide gel electrophoresis, or other separation techniques. DNA from the libraries or the separated DNA may be transferred to and immobilized on nitrocellulose or other suitable carrier material. In order to identify a clone or DNA which is homologous with SEQ ID NO: 1 or a subsequence thereof, the carrier material is used in a Southern blot.

For purposes of the present invention, hybridization indicates that the nucleotide sequence hybridizes to a labeled nucleic acid probe corresponding to the nucleotide sequence shown in SEQ ID NO: 1, its complementary strand, or a subsequence thereof, under very low to very high stringency conditions. Molecules to which the nucleic acid probe hybridizes under these conditions can be detected using X-ray film.

In a preferred aspect, the nucleic acid probe is nucleotides 106 to 1002 of SEQ ID NO: 1. In another preferred aspect, the nucleic acid probe is a polynucleotide sequence which encodes the polypeptide of SEQ ID NO: 2, or a subsequence thereof. In another preferred aspect, the nucleic acid probe is SEQ ID NO: 1. In another preferred aspect, the nucleic acid probe is the polynucleotide sequence contained in plasmid pOPHNN10787 which is contained in Escherichia coli DSM 17765, wherein the polynucleotide sequence thereof encodes a polypeptide having organophosphatse activity. In another preferred aspect, the nucleic acid probe is the mature polypeptide coding region contained in plasmid ppOPHNN10787.

In one aspect, the present invention relates to artificial variants comprising a conservative substitution, deletion, and/or insertion of one or more amino acids of SEQ ID NO: 2 or the mature polypeptide thereof. Preferably, amino acid changes are of a minor nature, that is conservative amino acid substitutions or insertions that do not significantly affect the folding and/or activity of the protein; small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine).

In addition to the 20 standard amino acids, non-standard amino acids (such as 4-hydroxyproline, 6-N-methyl lysine, 2-aminoisobutyric acid, isovaline, and alpha-methyl serine) may be substituted for amino acid residues of a wild-type polypeptide. A limited number of non-conservative amino acids, amino acids that are not encoded by the genetic code, and unnatural amino acids may be substituted for amino acid residues. “Unnatural amino acids” have been modified after protein synthesis, and/or have a chemical structure in their side chain(s) different from that of the standard amino acids. Unnatural amino acids can be chemically synthesized, and preferably, are commercially available, and include pipecolic acid, thiazolidine carboxylic acid, dehydroproline, 3- and 4-methylproline, and 3,3-dimethylproline.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in the parent polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for biological activity (i.e., OPH activity) to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309:59-64. The identities of essential amino acids can also be inferred from analysis of identities with polypeptides which are related to a polypeptide according to the invention.

Single or multiple amino acid substitutions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochem. 30:10832-10837; U.S. Pat. No. 5,223,409; WO 92/06204), and region-directed mutagenesis (Derbyshire et al., 1986, Gene 46:145; Ner et al., 1988, DNA 7:127).

Mutagenesis/shuffling methods can be combined with high-throughput, automated screening methods to detect activity of cloned, mutagenized polypeptides expressed by host cells. Mutagenized DNA molecules that encode active polypeptides can be recovered from the host cells and rapidly sequenced using standard methods in the art. These methods allow the rapid determination of the importance of individual amino acid residues in a polypeptide of interest, and can be applied to polypeptides of unknown structure.

The total number of amino acid substitutions, deletions and/or insertions of amino acids 36 to 334 of SEQ ID NO: 2 is 10, preferably 9, more preferably 8, more preferably 7, more preferably at most 6, more preferably at most 5, more preferably 4, even more preferably 3, most preferably 2, and even most preferably 1.

### Polynucleotides

The present invention also relates to isolated polynucleotides having a nucleotide sequence which encode a polypeptide of the present invention. In a preferred aspect, the nucleotide sequence is set forth in SEQ ID NO: 1. In another more preferred aspect, the nucleotide sequence is the sequence contained in plasmid pOPHNN10787 that is contained in Escherichia coli DSM 17765. In another preferred aspect, the nucleotide sequence is the mature polypeptide coding region of SEQ ID NO: 1. This region should at least comprise the nucleotides from position 106 to 1002 in SEQ ID NO: 1, but could also comprise the nucleotides from position 103 to 1002 in SEQ ID NO: 1. In another more preferred aspect, the nucleotide sequence is the mature polypeptide coding region contained in plasmid pOPHNN10787 that is contained in Escherichia coli DSM 17765. The present invention also encompasses nucleotide sequences which encode a polypeptide having the amino acid sequence of SEQ ID NO: 2 or the mature polypeptide thereof, which differs from SEQ ID NO: 1 by virtue of the degeneracy of the genetic code. The present invention also relates to subsequences of SEQ ID NO: 1 which encode fragments of SEQ ID NO: 2 that have OPH activity.

The present invention also relates to mutant polunucleotides comprising at least one mutation in the mature polypeptide coding sequence of SEQ ID NO: 1, in which the mutant nucleotide sequence encodes a polypeptide which comprises amino acids 36 to 334 of SEQ ID NO: 2.

The techniques used to isolate or clone a polynucleotide encoding a polypeptide are known in the art and include isolation from genomic DNA, preparation from cDNA, or a combination thereof. The cloning of the polynucleotides of the present invention from such genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligated activated transcription (LAT) and nucleotide sequence-based amplification (NASBA) may be used. The polynucleotides may be cloned from a strain of Ralstonia, or another or related organism and thus, for example, may be an allelic or species variant of the polypeptide encoding region of the nucleotide sequence.

The present invention also relates to polynucleotides having nucleotide sequences which have a degree of identity to the mature polypeptide coding sequence of SEQ ID NO: 1 of at least 80%, more preferably at least 81%, more preferably at least 82%, more preferably at least 84%, more preferably at least 86%, more preferably at least 88%, more preferably at least 90%, more preferably at least 92%, even more preferably at least 95%, and most preferably at least 97% identity, which encode a polypeptide having OPH activity. As explained herein the nucleotide sequence encoding the mature polypeptide starts at either position 103 or position 106 and ends at position 1002 of SEQ ID NO: 1.

Modification of a nucleotide sequence encoding a polypeptide of the present invention may be necessary for the synthesis of polypeptides substantially similar to the polypeptide. The term “substantially similar” to the polypeptide refers to non-naturally occurring forms of the polypeptide. These polypeptides may differ in some engineered way from the polypeptide isolated from its native source, e.g., artificial variants that differ in specific activity, thermostability, pH optimum, or the like. The variant sequence may be constructed on the basis of the nucleotide sequence presented as the polypeptide encoding region of SEQ ID NO: 1, e.g., a subsequence thereof, and/or by introduction of nucleotide substitutions which do not give rise to another amino acid sequence of the polypeptide encoded by the nucleotide sequence, but which correspond to the codon usage of the host organism intended for production of the enzyme, or by introduction of nucleotide substitutions which may give rise to a different amino acid sequence. For a general description of nucleotide substitution, see, e.g. Ford et al., 1991, Protein Expression and Purification 2: 95-107.

It will be apparent to those skilled in the art that such substitutions can be made outside the regions critical to the function of the molecule and still result in an active polypeptide. Amino acid residues essential to the activity of the polypeptide encoded by an isolated polynucleotide of the invention, and therefore preferably not subject to substitution, may be identified according to procedures known in the art, such as site-directed mutagenesis or alanine-scanning mutagenesis (see, e.g., Cunningham and Wells, 1989, Science 244: 1081-1085). In the latter technique, mutations are introduced at every positively charged residue in the molecule, and the resultant mutant molecules are tested for OPH activity to identify amino acid residues that are critical to the activity of the molecule. Sites of substrate-enzyme interaction can also be determined by analysis of the three-dimensional structure as determined by such techniques as nuclear magnetic resonance analysis, crystallography or photoaffinity labelling (see, e.g., de Vos et al., 1992, Science 255: 306-312; Smith et al., 1992, Journal of Molecular Biology 224: 899-904; Wlodaver et al., 1992, FEBS Letters 309: 59-64).

The present invention also relates to isolated polynucleotides encoding a polypeptide of the present invention, which hybridize under very low stringency conditions, preferably low stringency conditions, more preferably medium stringency conditions, more preferably medium-high stringency conditions, even more preferably high stringency conditions, and most preferably very high stringency conditions with (i) nucleotides 106 to 1002 of SEQ ID NO: 1, (ii) a subsequence of (i) of at least 100 nucleotides, or (iii) a complementary strand of (i) or (ii); and allelic variants and subsequences thereof (Sambrook et al., 1989, supra), as defined herein.

### Nucleic Acid Constructs

The present invention also relates to nucleic acid constructs comprising an isolated polynucleotide of the present invention operably linked to one or more control sequences which direct the expression of the coding sequence in a suitable host cell under conditions compatible with the control sequences.

An isolated polynucleotide encoding a polypeptide of the present invention may be manipulated in a variety of ways to provide for expression of the polypeptide. Manipulation of the polynucleotide's sequence prior to its insertion into a vector may be desirable or necessary depending on the expression vector. The techniques for modifying polynucleotide sequences utilizing recombinant DNA methods are well known in the art.

The control sequence may be an appropriate promoter sequence, a nucleotide sequence which is recognized by a host cell for expression of a polynucleotide encoding a polypeptide of the present invention. The promoter sequence contains transcriptional control sequences which mediate the expression of the polypeptide. The promoter may be any nucleotide sequence which shows transcriptional activity in the host cell of choice including mutant, truncated, and hybrid promoters, and may be obtained from genes encoding extracellular or intracellular polypeptides either homologous or heterologous to the host cell.

Examples of suitable promoters for directing the transcription of the nucleic acid constructs of the present invention, especially in a bacterial host cell, are the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (Villa-Kamaroff et al., 1978, Proceedings of the National Academy of Sciences USA 75: 3727-3731), as well as the tac promoter (DeBoer et al., 1983, Proceedings of the National Academy of Sciences USA 80: 21-25). Further promoters are described in “Useful proteins from recombinant bacteria” in Scientific American, 1980, 242: 74-94; and in Sambrook et al., 1989, supra.

Examples of suitable promoters for directing the transcription of the nucleic acid constructs of the present invention in a filamentous fungal host cell are promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, Fusarium venenatum amyloglucosidase (WO 00/56900), Fusarium venenatum Daria (WO 00/56900), Fusarium venenatum Quinn (WO 00/56900), Fusarium oxysporum trypsin-like protease (WO 96/00787), Trichoderma reesei beta-glucosidase, Trichoderma reesei cellobiohydrolase I, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a hybrid of the promoters from the genes for Aspergillus niger neutral alpha-amylase and Aspergillus oryzae triose phosphate isomerase); and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionine (CUP1), and Saccharomyces cerevisiae 3-phosphoglycerate kinase. Other useful promoters for yeast host cells are described by Romanos et al., 1992, Yeast 8: 423-488.

The control sequence may also be a suitable transcription terminator sequence, a sequence recognized by a host cell to terminate transcription. The terminator sequence is operably linked to the 3′ terminus of the nucleotide sequence encoding the polypeptide. Any terminator which is functional in the host cell of choice may be used in the present invention.

Preferred terminators for filamentous fungal host cells are obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be a suitable leader sequence, a nontranslated region of an mRNA which is important for translation by the host cell. The leader sequence is operably linked to the 5′ terminus of the nucleotide sequence encoding the polypeptide. Any leader sequence that is functional in the host cell of choice may be used in the present invention.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′ terminus of the nucleotide sequence and which, when transcribed, is recognized by the host cell as a signal to add polyadenosine residues to transcribed mRNA. Any polyadenylation sequence which is functional in the host cell of choice may be used in the present invention.

Preferred polyadenylation sequences for filamentous fungal host cells are obtained from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase.

Useful polyadenylation sequences for yeast host cells are described by Guo and Sherman, 1995, Molecular Cellular Biology 15: 5983-5990.

The control sequence may also be a signal peptide coding region that codes for an amino acid sequence linked to the amino terminus of a polypeptide and directs the encoded polypeptide into the cell's secretory pathway. The 5′ end of the coding sequence of the nucleotide sequence may inherently contain a signal peptide coding region naturally linked in translation reading frame with the segment of the coding region which encodes the secreted polypeptide. Alternatively, the 5′ end of the coding sequence may contain a signal peptide coding region which is foreign to the coding sequence. The foreign signal peptide coding region may be required where the coding sequence does not naturally contain a signal peptide coding region. Alternatively, the foreign signal peptide coding region may simply replace the natural signal peptide coding region in order to enhance secretion of the polypeptide. However, any signal peptide coding region which directs the expressed polypeptide into the secretory pathway of a host cell of choice may be used in the present invention.

Effective signal peptide coding regions for bacterial host cells are the signal peptide coding regions obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding regions for filamentous fungal host cells are the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase.

The OPH of the present invention as shown in SEQ ID NO 2 and encoded for by SEQ ID NO 1 comprises its natural signal peptide coding region. This native signal peptide consists of amino acids 1 to 34 and maybe even 35 of SEQ ID NO 2, encoded for by nucleotides 1 to 102 or 105 in SEQ ID NO 1. Since the origin of the OPH according to the invention is bacterial the native signal peptide is particularly useful for expression of the OPH in a bacterial host cell. However, as evidenced by the data presented in the examples, this native bacterial signal peptide will also result in secretion of the OPH when expressed in a fungal host cell.

Useful signal peptides for yeast host cells are obtained from the genes for Saccharomyces cerevisiae alpha-factor and Saccharomyces cerevisiae invertase. Other useful signal peptide coding regions are described by Romanos et al., 1992, supra.

The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila laccase (WO 95/33836).

Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the TAKA alpha-amylase promoter, Aspergillus niger glucoamylase promoter, and Aspergillus oryzae glucoamylase promoter may be used as regulatory sequences. Other examples of regulatory sequences are those which allow for gene amplification. In eukaryotic systems, these include the dihydrofolate reductase gene which is amplified in the presence of methotrexate, and the metallothionein genes which are amplified with heavy metals. In these cases, the nucleotide sequence encoding the polypeptide would be operably linked with the regulatory sequence.

### Expression Vectors

The present invention also relates to recombinant expression vectors comprising a polynucleotide of the present invention, a promoter, and transcriptional and translational stop signals. The various nucleic acids and control sequences described above may be joined together to produce a recombinant expression vector which may include one or more convenient restriction sites to allow for insertion or substitution of the nucleotide sequence encoding the polypeptide at such sites. Alternatively, a nucleotide sequence of the present invention may be expressed by inserting the nucleotide sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) which can be conveniently subjected to recombinant DNA procedures and can bring about expression of the nucleotide sequence. The choice of the vector will typically depend on the compatibility of the vector with the host cell into which the vector is to be introduced. The vectors may be linear or closed circular plasmids.

The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The vectors of the present invention preferably contain one or more selectable markers which permit easy selection of transformed cells. A selectable marker is a gene the product of which provides for biocide or viral resistance, resistance to heavy metals, prototrophy to auxotrophs, and the like.

A conditionally essential gene may function as a non-antibiotic selectable marker. Non-limiting examples of bacterial conditionally essential non-antibiotic selectable markers are the dal genes from Bacillus subtilis, Bacillus licheniformis, or other Bacilli, that are only essential when the bacterium is cultivated in the absence of D-alanine. Also the genes encoding enzymes involved in the turnover of UDP-galactose can function as conditionally essential markers in a cell when the cell is grown in the presence of galactose or grown in a medium which gives rise to the presence of galactose. Non-limiting examples of such genes are those from B. subtilis or B. licheniformis encoding UTP-dependent phosphorylase (EC 2.7.7.10), UDP-glucose-dependent uridylyltransferase (EC 2.7.7.12), or UDP-galactose epimerase (EC 5.1.3.2). Also a xylose isomerase gene such as xylA, of Bacilli can be used as selectable markers in cells grown in minimal medium with xylose as sole carbon source. The genes necessary for utilizing gluconate, gntK, and gntP can also be used as selectable markers in cells grown in minimal medium with gluconate as sole carbon source. Other examples of conditionally essential genes are known in the art. Antibiotic selectable markers confer antibiotic resistance to such antibiotics as ampicillin, kanamycin, chloramphenicol, erythromycin, tetracycline, neomycin, hygromycin or methotrexate.

Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are the amdS and pyrG genes of Aspergillus nidulans or Aspergillus oryzae and the bar gene of Streptomyces hygroscopicus.

The vectors of the present invention preferably contain an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or nonhomologous recombination. Alternatively, the vector may contain additional nucleotide sequences for directing integration by homologous recombination into the genome of the host cell at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which have a high degree of identity with the corresponding target sequence to enhance the probability of homologous recombination. The integrational elements may be any sequence that is homologous with the target sequence in the genome of the host cell. Furthermore, the integrational elements may be non-encoding or encoding nucleotide sequences. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication which functions in a cell. The term “origin of replication” or “plasmid replicator” is defined herein as a nucleotide sequence that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98:61-67; Cullen et al., 1987, Nucleic Acids Research 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into the host cell to increase production of the gene product. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention, which are advantageously used in the recombinant production of the polypeptides. A vector comprising a polynucleotide of the present invention is introduced into a host cell so that the vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be a prokaryote or a eukaryote. The host cell may be unicellular.

Useful unicellular microorganisms are bacterial cells such as gram positive bacteria including, but not limited to, a Bacillus cell, e.g., Bacillus alkalophilus, Bacillus amyloliquefaciens, Bacillus brevis, Bacillus circulans, Bacillus clausii, Bacillus coagulans, Bacillus lautus, Bacillus lentus, Bacillus licheniformis, Bacillus megaterium, Bacillus stearothermophilus, Bacillus subtilis, and Bacillus thuringiensis; or a Streptomyces cell, e.g., Streptomyces lividans and Streptomyces murinus, or gram negative bacteria such as E. coli and Pseudomonas sp. In a preferred aspect, the bacterial host cell is a Bacillus lentus, Bacillus licheniformis, Bacillus stearothermophilus, or Bacillus subtilis cell. In another preferred aspect, the Bacillus cell is an alkalophilic Bacillus.

The introduction of a vector into a bacterial host cell may, for instance, be effected by protoplast transformation (see, e.g., Chang and Cohen, 1979, Molecular General Genetics 168: 111-115), using competent cells (see, e.g., Young and Spizizin, 1961, Journal of Bacteriology 81: 823-829, or Dubnau and Davidoff-Abelson, 1971, Journal of Molecular Biology 56: 209-221), electroporation (see, e.g., Shigekawa and Dower, 1988, Biotechniques 6: 742-751), or conjugation (see, e.g., Koehler and Thorne, 1987, Journal of Bacteriology 169: 5771-5278).

The host cell may also be a eukaryote, such as a mammalian, insect, plant, or fungal cell.

In a preferred aspect, the host cell is a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

In a more preferred aspect, the fungal host cell is a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980).

In an even more preferred aspect, the yeast host cell is a Candida, Hansenula, Kluyveromyces, Pichia, Saccharomyces, Schizosaccharomyces, or Yarrowia cell.

In a most preferred aspect, the yeast host cell is a Saccharomyces carlsbergensis, Saccharomyces cerevisiae, Saccharomyces diastaticus, Saccharomyces douglasii, Saccharomyces kluyveri, Saccharomyces norbensis or Saccharomyces oviformis cell. In another most preferred aspect, the yeast host cell is a Kluyveromyces lactis cell. In another most preferred aspect, the yeast host cell is a Yarrowia lipolytica cell.

In another more preferred aspect, the fungal host cell is a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

In an even more preferred aspect, the filamentous fungal host cell is an Acremonium, Aspergillus, Aureobasidium, Bjerkandera, Ceriporiopsis, Coprinus, Coriolus, Cryptococcus, Filobasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

In a most preferred aspect, the filamentous fungal host cell is an Aspergillus awamori, Aspergillus fumigatus, Aspergillus foetidus, Aspergillus japonicus, Aspergillus nidulans, Aspergillus niger or Aspergillus oryzae cell. In another most preferred aspect, the filamentous fungal host cell is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioldes, or Fusarium venenatum cell. In another most preferred aspect, the filamentous fungal host cell is a Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, or Ceriporiopsis subvermispora, Coprinus cinereus, Coriolus hirsutus, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238 023 and Yelton et al., 1984, Proceedings of the National Academy of Sciences USA 81: 1470-1474. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

### Plants

The present invention also relates to a transgenic plant, plant part, or plant cell which has been transformed with a nucleotide sequence encoding a polypeptide having OPH activity of the present invention so as to express and produce the polypeptide in recoverable quantities. The polypeptide may be recovered from the plant or plant part.

Plant organisms for the purposes of the invention are organisms capable of being photosynthetically active such as, for example, algae, cyanobacteria and mosses. Preferred algae are green algae such as, for example, algae from the genus Haematococcus, Phaedactylum tricornatum, Volvox or Dunaliella.

The transgenic plant can be dicotyledonous (a dicot) or monocotyledonous (a monocot). Examples of monocot plants are grasses, such as meadow grass (blue grass, Poa), forage grass such as Festuca, Lolium, temperate grass, such as Agrostis, and cereals, e.g., wheat, oats, rye, barley, rice, sorghum, and maize (corn).

Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana.

Examples of plant parts are stem, callus, leaves, root, fruits, seeds, and tubers as well as the individual tissues comprising these parts, e.g., epidermis, mesophyll, parenchyme, vascular tissues, meristems. Specific plant cell compartments, such as chloroplasts, apoplasts, mitochondria, vacuoles, peroxisomes and cytoplasm are also considered to be a plant part. Furthermore, any plant cell, whatever the tissue origin, is considered to be a plant part. Likewise, plant parts such as specific tissues and cells isolated to facilitate the utilisation of the invention are also considered plant parts, e.g., embryos, endosperms, aleurone and seeds coats.

Also included within the scope of the present invention are the progeny of such plants, plant parts, and plant cells.

The transgenic plant or plant cell expressing a polypeptide of the present invention may be constructed in accordance with methods known in the art. In short, the plant or plant cell is constructed by incorporating one or more expression constructs encoding a polypeptide of the present invention into the plant host genome and propagating the resulting modified plant or plant cell into a transgenic plant or plant cell.

The expression construct is conveniently a nucleic acid construct which comprises a polynucleotide encoding a polypeptide of the present invention operably linked with appropriate regulatory sequences required for expression of the nucleotide sequence in the plant or plant part of choice. Furthermore, the expression construct may comprise a selectable marker useful for identifying host cells into which the expression construct has been integrated and DNA sequences necessary for introduction of the construct into the plant in question (the latter depends on the DNA introduction method to be used).

Cloning vectors and techniques for the genetic manipulation of ciliates and algae are known to the skilled worker (WO 98/01572; Falciatore et al. (1999) Marine Biotechnology 1 (3): 239-251; Dunahay et al. (1995) J Phycol 31: 10004-1012; Mayfield et al., 2003, PNAS 100 (2): 438-442).

The choice of regulatory sequences, such as promoter and terminator sequences and optionally signal or transit sequences are determined, for example, on the basis of when, where, and how the polypeptide is desired to be expressed. For instance, the expression of the gene encoding a polypeptide of the present invention may be constitutive or inducible, or may be developmental, stage or tissue specific, and the gene product may be targeted to a specific tissue or plant part such as seeds or leaves. Regulatory sequences are, for example, described by Tague et al., 1988, Plant Physiology 86: 506.

For constitutive expression, the 35S-CaMV, the maize ubiquitin 1, and the rice actin 1 promoter may be used (Franck et al., 1980, Cell 21: 285-294, Christensen et al., 1992, Plant Mo. Biol. 18: 675-689; Zhang et al., 1991, Plant Cell 3: 1155-1165). Organ-specific promoters may be, for example, a promoter from storage sink tissues such as seeds, potato tubers, and fruits (Edwards & Coruzzi, 1990, Ann. Rev. Genet. 24: 275-303), or from metabolic sink tissues such as meristems (Ito et al., 1994, Plant Mol. Biol. 24: 863-878), a seed specific promoter such as the glutelin, prolamin, globulin, or albumin promoter from rice (Wu et al., 1998, Plant and Cell Physiology 39: 885-889), a Vicia faba promoter from the legumin B4 and the unknown seed protein gene from Vicia faba (Conrad et al., 1998, Journal of Plant Physiology 152: 708-711), a promoter from a seed oil body protein (Chen et al., 1998, Plant and Cell Physiology 39: 935-941), the storage protein napA promoter from Brassica napus, or any other seed specific promoter known in the art, e.g., as described in WO 91/14772. Furthermore, the promoter may be a leaf specific promoter such as the rbcs promoter from rice or tomato (Kyozuka et al., 1993, Plant Physiology 102: 991-1000, the chlorella virus adenine methyltransferase gene promoter (Mitra and Higgins, 1994, Plant Molecular Biology 26: 85-93), or the aldP gene promoter from rice (Kagaya et al., 1995, Molecular and General Genetics 248: 668-674), or a wound inducible promoter such as the potato pin2 promoter (Xu et al., 1993, Plant Molecular Biology 22: 573-588). Likewise, the promoter may inducible by abiotic treatments such as temperature, drought, or alterations in salinity or induced by exogenously applied substances that activate the promoter, e.g., ethanol, oestrogens, plant hormones such as ethylene, abscisic acid, and gibberellic acid, and heavy metals.

A promoter enhancer element may also be used to achieve higher expression of a polypeptide of the present invention in the plant. For instance, the promoter enhancer element may be an intron which is placed between the promoter and the nucleotide sequence encoding a polypeptide of the present invention. For instance, Xu et al., 1993, supra, disclose the use of the first intron of the rice actin 1 gene to enhance expression.

The selectable marker gene and any other parts of the expression construct may be chosen from those available in the art.

The nucleic acid construct is incorporated into the plant genome according to conventional techniques known in the art, including Agrobacterium-mediated transformation, virus-mediated transformation, microinjection, particle bombardment, biolistic transformation, and electroporation (Gasser et al., 1990, Science 244: 1293; Potrykus, 1990, Bio/Technology 8: 535; Shimamoto et al., 1989, Nature 338: 274).

Presently, Agrobacterium tumefaciens-mediated gene transfer is the method of choice for generating transgenic dicots (for a review, see Hooykas and Schilperoort, 1992, Plant Molecular Biology 19: 15-38) and can also be used for transforming monocots, although other transformation methods are often used for these plants. Presently, the method of choice for generating transgenic monocots is particle bombardment (microscopic gold or tungsten particles coated with the transforming DNA) of embryonic calli or developing embryos (Christou, 1992, Plant Journal 2: 275-281; Shimamoto, 1994, Current Opinion Biotechnology 5: 158-162; Vasil et al., 1992, Bio/Technology 10: 667-674). An alternative method for transformation of monocots is based on protoplast transformation as described by Omirulleh etal., 1993, Plant MolecularBiology 21: 415-428.

Following transformation, the transformants having incorporated the expression construct are selected and regenerated into whole plants according to methods well-known in the art. Often the transformation procedure is designed for the selective elimination of selection genes either during regeneration or in the following generations by using, for example, co-transformation with two separate T-DNA constructs or site specific excision of the selection gene by a specific recombinase.

The present invention also relates to methods for producing a polypeptide of the present invention comprising (a) cultivating a transgenic plant or a plant cell comprising a polynucleotide encoding a polypeptide having organophosphatase activity of the present invention under conditions conducive for production of the polypeptide; and (b) recovering the polypeptide.

In a further embodiment the present invention relates to a method for producing the polypeptide of the invention, comprising (a) cultivating an algae comprising a polynucleotide encoding a polypeptide having organophosphatase activity of the present invention under conditions conducive for production of the polypeptide; and (b) recovering the polypeptide.

### Methods of Production

The present invention also relates to methods for producing a polypeptide of the present invention, comprising (a) cultivating a cell, which in its wild-type form is capable of producing the polypeptide, under conditions conducive for production of the polypeptide; and (b) recovering the polypeptide. Preferably, the cell is of the genus Ralstonia, and more preferably Ralstonia pickettii.

The present invention also relates to methods for producing a polypeptide of the present invention, comprising (a) cultivating a host cell comprising a nucleic acid construct comprising a nucleotide sequence encoding the polypeptide of the invention under conditions conducive for production of the polypeptide; and (b) recovering the polypeptide.

The present invention also relates to methods for producing a polypeptide of the present invention, comprising (a) cultivating a host cell under conditions conducive for production of the polypeptide, wherein the host cell comprises a mutant nucleotide sequence having at least one mutation in the mature polypeptide coding region of SEQ ID NO: 1, wherein the mutant nucleotide sequence encodes a polypeptide which comprises amino acids 36 to 334 of SEQ ID NO: 2, and (b) recovering the polypeptide.

In the production methods of the present invention, the cells are cultivated in a nutrient medium suitable for production of the polypeptide using methods well known in the art. For example, the cell may be cultivated by shake flask cultivation, and small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptides may be detected using methods known in the art that are specific for the polypeptides. These detection methods may include use of specific antibodies, formation of an enzyme product, or disappearance of an enzyme substrate. For example, an enzyme assay may be used to determine the activity of the polypeptide as described herein.

The resulting polypeptide may be recovered using methods known in the art. For example, the polypeptide may be recovered from the nutrient medium by conventional procedures including, but not limited to, centrifugation, filtration, extraction, spray-drying, evaporation, or precipitation.

The polypeptides of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989).

### Compositions

The present invention also relates to compositions comprising a polypeptide of the present invention. Preferably, the compositions are enriched in such a polypeptide. The term “enriched” indicates that the OPH activity of the composition has been increased, e.g. with an enrichment factor of 1.1.

The composition may comprise a polypeptide of the present invention as the major enzymatic component, e.g., a mono-component composition. Alternatively, the composition may comprise multiple enzymatic activities, such as an aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, lipase, mannosidase, oxidase, pectinolytic enzyme, peptidoglutaminase, peroxidase, phytase, polyphenoloxidase, proteolytic enzyme, ribonuclease, transglutaminase, urease, or xylanase. The additional enzyme(s) may be produced, for example, by a microorganism belonging to the genus Aspergillus, preferably Aspergillus aculeatus, Aspergillus awamori, Aspergillus fumigatus, Aspergillus foetidus, Aspergillus japonicus, Aspergillus nidulans, Aspergillus niger, or Aspergillus oryzae; Fusarium, preferably Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sulphureum, Fusarium toruloseum, Fusarium trichothecioides, or Fusarium venenatum; Humicola, preferably Humicola insolens or Humicola lanuginosa; or Trichoderma, preferably Trichoderma harzianum, Trichoderma koningii, Trichoderma longibrachiatum, Trichoderma reesei, or Trichoderma viride.

In a particular embodiment the invention relates to a composition for hydrolysing an organophosphatase molecule, said composition comprising a polypeptide according to the invention and one or more acceptable carriers.

In another particular embodiment the composition comprises a host cell expressing the organophosphatase of the invention.

The polypeptide compositions may be prepared in accordance with methods known in the art and may be in the form of a liquid or a dry composition. For instance, the polypeptide composition may be in the form of a granulate or a microgranulate. The polypeptide to be included in the composition may be stabilized in accordance with methods known in the art.

Examples are given below of preferred uses of the polypeptide compositions of the invention. The dosage of the polypeptide composition of the invention and other conditions under which the composition is used may be determined on the basis of methods known in the art.

### Uses

The present invention is also directed to methods for using the polypeptides having organophosphatase activity for hydrolysing an organophosphorus molecule by contacting the said molecule with the organophosphatase.

Exposing or contacting the the organophosphorus molecule to the organophasphatase according to the invention can be performed in many ways, e.g. by exposing a composition or a host cell comprising the OPH to the said molecule.

The present invention is further described by the following examples which should not be construed as limiting the scope of the invention.

### EXAMPLES

Chemicals used as buffers and substrates were commercial products of at least reagent grade.

### Example 1

### Cloning and Expression of OPH from Ralstonia picketii ATCC27511

Reagents and media

| | | | |
|---|---|---|---|
| Nutrient agar | Peptone | 5.0 | g |
| | Meat extract | 3.0 | g |
| | Agar | 15.0 | g |
| | Distilled water | 1000.0 | ml |

Adjust pH to 7.0.
Autoclave at 121 ° C., 16 minutes

| | | | |
|---|---|---|---|
| Nutrient broth: | Peptone | 5.0 | g |
| | Meat extract | 3.0 | g |
| | Distilled water | 1000.0 | ml |

Adjust pH to 7.0.
Autoclave at 121° C., 16 minutes

| | |
|---|---|
| TE | 10 mM Tris-HCl, pH 7.4 |
| | 1 mM EDTA, pH 8.0 |
| TEL | 50 mg/ml Lysozym in TE-buffer |
| Thiocyanate | 5M guanidium thiocyanate |
| | 100 mM EDTA |
| | 0.6% w/v N-laurylsarcosine, sodium salt |
| | 60 g thiocyanate, 20 ml 0.5 M EDTA, pH |
| | 8.0, 20 ml H₂O dissolves at 65° C. |

Cool down to room temperature (RT) and add 0.6 g
N-laurylsarcosine. Add H₂O to 100 ml and
filter it through a 0.2 μ sterile filter.

| | | | |
|---|---|---|---|
| NH₄Ac | 7.5 M CH₃COONH₄ | | |
| TER | 1 μg/ml RNAse A in TE-buffer | | |
| CIA | Chloroform/isoamyl alcohol 24:1 | | |
| TY*2 medium | Tryptone | 40 | g |
| | Yeast Extract | 10 | g |
| | 1% Ferrochloride | 1.4 | ml |
| | 1% Mangan(II)-chloride | 0.2 | ml |
| | 1% Magnesiumsulfate | 3.0 | ml |

Add distilled water up to 1000 ml; adjust pH to 7.3 and
autoclave at 121° C., 16 minutes.

| | | | |
|---|---|---|---|
| Lysisbuffer: | HEPES 100 mM pH 7 | 200 | μl |
| | Triton X-100 | 20 | μl |
| | DNA\acute{ }se 10 mg/ml | 2 | μl |
| | Lysozym 10 mg/ml | 2 | μl |

Add distilled water up to 1 ml

| | |
|---|---|
| DNA\acute{ }se | 10 mg/ml in 50% glycerol an 10 mM |
| | Hepes pH 7. |

### Cloning of SEQ ID NO: 1

SEQ ID NO: 1 is the DNA sequence encoding the OPH from Ralstonia picketii ATCC27511.
Genomic DNA from Ralstonia picketii can be isolated according to the following procedure from an over night culture in nutrient broth at 37° C.:
1. Harvest 1.5 ml culture and resuspend in 100 μl TEL. Incubate at 37° C. for 30 min.
2. Add 500 μl thiocynate buffer and leave at room temperature for 10 min.
3. Add 250 μl NH₄Ac and leave at ice for 10 min.
4. Add 500 μl CIA and mix.
5. Transfer to a microcentrifuge and spin for 10 min. at full speed.
6. Transfer supernatant to a new Eppendorf tube and add 0.54 volume cold isopropanol. Mix thoroughly.
7. Spin and wash the DNA pellet with 70% EtOH.
8. Resuspend the genomic DNA in 100 μl TER.
   The genomic DNA from Ralstonia picketii ATCC27511 can be used as template for PCR amplification of the OPH Ralstonia picketii ATCC27511 gene by standard PCR methods using primer 1918 and primer 1919.

### Primer 1918 (SEQ ID NO 3):

5′-GGATTAATGCGTTTGCTTACTTCTGTCGCCGTG -3′, start codon in bold and restriction site Ase I is underlined

### Primer 1919 (SEQ ID NO 4):

5′-CCGCTCGAGATCAGCGCGGTCTGTGCGCAG-3′, restriction site Xho I is underlined.
   Please note that using primer 1919 the stop codon TGA is removed, useful for expression with C-terminal His-tag's.
   The PCR product amplified with primer 1918 and 1919 was digested with restriction enzyme Ase I and Xho I using standard method, and the resulting fragment was ligated to Nde I/Xho I digested pET-30a(+) (Novagen) vector prior to transformation into E. coli DH10B. E. coli DH10B Kanamycin resistant transformants were further analyzed by DNA sequencing of the OPH gene insert in the pET-30a(+). A plasmid with correct sequence, pOPHNN10787, was selected.

### Fermentation

pOPHNN10787 was transformed into E. coli BL21 according to standard transformation protocols. A correct transformant, E. coli BL21 (pOPHNN10787), was selected by ampicillin selection.
Recombinant expression of the His tag OPH from Ralstonia picketii ATCC27511 was done from E. coli BL21 (pOPHNN10787) according to standard protocols (see e.g. the pET System Manual).

### Lysis of Cells

Cells were harvested at 4000 rpm, 30 min. Cell pellet was resuspended in lysis buffer (a 1/10 of the culture volume) and transferred to 37° C. under shaking (250 rpm) for 15 minutes. The samples were centrifuged (4000 rpm, 10 minutes) and the supernatants were collected. The level of expression was analysed by standard SDS PAGE analysis (no visible band) and the supernatants were used for the paraoxonase assay as described in Example 2.

### Example 2

### Expression and Purification of OPH from Aspergillus oryzae

Reagent and media
Trace element mixture (100 × concentrate)

| | | |
|---|---|---|
| MgCl₂—6H₂O | 12.5 | g |
| CaCl2 | 0.55 | g |
| FeCl₃—6H₂O | 1.35 | g |
| MnCl₂—4H₂O | 0.1 | g |
| ZnCl₂ | 0.17 | g |
| CuCl₂—2H₂O | 0.043 | g |
| CoCl₂—6H₂O | 0.06 | g |
| Na₂MoO₄—2H₂O | 0.06 | g |

YP medium

| | | |
|---|---|---|
| Yeast extract | 10 | g |
| Peptone | 20 | g |
| Ion exchanged water to 1 L | | |

Make sure pH is 6.8 as expected, then autoclave medium

### Construction of Plasmid

Plasmid DNA of pOPHNN10787 can be prepared by standard methods. The obtained plasmid can then be used as template in a standard PCR reaction using either primer 106 and 107 or primer 121 and 107.

### Primer 106 (SEQ ID NO 5):

5′-GTA GGA TCC ACC ATG CGT TTG CTT ACT TCT GTC -3′, start codon in bold and restriction site BamHI underlined.

### Primer 107 (SEQ ID NO 6):

5′-CTT CTT AAG TCA ATC AGC GCG GTC TGT GC -3′, stop codon in bold and restriction site AfIII underlined.

### Primer 121 (SEQ ID NO 7):

5′-GGG GAT CCA CCA TGA GAT TAT CGA CTT CGA GTC TCT TCC TTT CCG TGT CTC TGC TGG GGA AGC TGG CCC TCG GGC AAA CTG CAA CTT CAG CCG C -3′, start codon in bold and restriction site BamHI underlined.
   Please note that primer 106 maintains the native bacterial signal sequence, while primer 121 replaces the native signal with the signal sequence from the fungal amylase SP288 (the secretion signal from the acid amylase SP288 from A. niger).
   The PCR products were digested with restriction enzymes BamHI and AfIII using standard methods. The resulting fragments were ligated to BamHI/AfIII digested pEN12516 vector (pENI2516 is described in WO 2004/069872 Example 2) prior to transformation into E. coli DH10B. Plasmids with the correct sequence, pLAQ055 and pLAQ062, were selected. The verified constructs were transformed into protoplasts of Aspergillus oryzae strain ToC1512 (ToC1512 is described in WO2005/070962, Example 11).

### Fermentation

The transformants were grown in YP medium added 2% maltose and trace elements for 4-5 days at 34° C. The level of expression was analysed by standard SDS PAGE analysis, which showed a strong band of the correct size without any apparent degradation for both expression strains. The enzymatic activity was analysed using the EnzCheck® Paraoxonase Assay Kit (Molecular Probes™, Cat # E33702), which showed high levels of paraoxonase activity in the growth media of both expression strains. The protein band was blotted onto a membrane and its N-terminal sequence was determined using standard methods. Two N-terminal amino acid sequences were determined: ATQQRTQ and TQQRTQV. The identity of the protein was thus confirmed and it appears that even the native signal sequence is processed correctly by A. oryzae. The result of the determination of the N-terminal demonstrates two possible positions as the start of the mature enzyme at position 35 and 36 in SEQ ID NO: 2 respectively.

### Purification

Growth medium from a standard fermentation was sterile filtered using a 0.45 μm membrane filter. The pH of the growth media was then adjusted to pH 5.8 prior to binding onto a column with SP-sepharose pre-equilibrated with 50 mM acetate buffer, pH 5.8. The column was then washed with the same buffer before eluting the protein with a step-wise gradient of NaCl.

### Activitv Assay

The enzymatic activity was determined using the EnzChek® Paraoxonase assay from Molecular Probes™. Paraoxonase has multiple activities including organophosphatase and phosphotriesterase. The organophosphatase activity confers protection against toxic organophosphates such as insecticides, which are a common source of chemical intolerance, and nerve agents such as sarin and VX. The assay was employed to determine the level of expression obtained by fermenting the already described genetically modified organisms.
The results clearly indicate that expression in A. oryzae is superior to all other tested host organisms. A further advantage is that the enzyme is secreted to the growth media from this organism.

| | Rel. |
|---|---|
| Organism | activity |
| Blank | 0.00 |
| E. coli, intracellular | 0.25 |
| A. oryzae (native signal), extracellular | 18.00 |
| A. oryzae (SP288 signal), extracellular | 100.00 |

Experiments to identify the cofactor of the enzyme have also been carried out. Several small scale fermentations were set up, where only one of the individual components present in the trace element mixture was added. The fermentation added only Mn resulted in the highest level of activity, which indicates that Mn is the preferred cofactor of the enzyme.

| | Rel. |
|---|---|
| Added cofactor | activity |
| Blank | 0 |
| Trace element mix | 53 |
| MgCl₂ | 11 |
| CaCl₂ | 5 |
| FeCl₃ | 8 |
| MnCl₂ | 100 |

1. An isolated polypeptide having organophosphatase activity, selected from the group consisting of:
(a) a polypeptide comprising an amino acid sequence which has at least 87% identity with amino acids 36 to 334 of SEQ ID NO: 2;
(b) a polypeptide which is encoded by a polynucleotide which hybridizes under at least medium stringency conditions with (i) nucleotides 106 to 1002 of SEQ ID NO: 1, or (ii) a complementary strand of (i);
(c) a variant comprising a conservative substitution, deletion, and/or insertion of one or more amino acids of amino acids 36 to 334 of SEQ ID NO: 2.

2. The polypeptide of claim 1, comprising an amino acid sequence which has at least 88% identity with amino acids 36 to 334 of SEQ ID NO: 2.

3. The polypeptide of claim 2, comprising an amino acid sequence which has at least 90% identity with amino acids 36 to 334 of SEQ ID NO: 2.

4. The polypeptide of claim 3, comprising an amino acid sequence which has at least 95% identity with amino acids 36 to 334 of SEQ ID NO: 2.

5. An isolated polypeptide having organophosphatase activity comprising the amino acids 36 to 334 of SEQ ID NO: 2.

6. The polypeptide of claim 1, which consists of amino acids 35 to 334 of SEQ ID NO: 2 or a fragment thereof having organophosphatase activity.

7. An isolated polypeptide having organophosphatase activity, which consists of SEQ ID NO: 2.

8. The polypeptide of claim 1, which is encoded by the polynucleotide contained in plasmid pOPHNN10787 which is contained in E. coli DSM 17765.

9. The isolated polypeptide according to claim 1, comprising the cofactor Mn.

10. An isolated polynucleotide encoding an organophosphatase, selected from the group consisting of:
(a) a nucleic acid sequence encoding a polypeptide comprising an amino acid sequence which has at least 87% identity with amino acids 36 to 334 of SEQ ID NO: 2;
(b) a nucleic acid sequence having at least 81% identity with the nucleic acid sequence from position 106 to 1002 of SEQ ID NO: 1;
(c) a nucleic acid sequence which hybridizes under medium stringency conditions with (i) the nucleic acid sequence from position 106 to 1002 of SEQ ID NO: 1, (ii) a subsequence of (i) of at least 100 nucleotides, or (iii) a complementary strand of (i) or (ii);
(d) an allelic variant of (a), (b), (c);
(e) a subsequence of (a), (b), (c), or (d), wherein the subsequence encodes a polypeptide fragment which has organophosphatase activity.

11. The isolated polynucleotide of claim 10, selected from the group consisting of: (a) a nucleic acid sequences encoding a polypeptide comprising the amino acid sequence of SEQ ID NO: 2; (b) has the nucleic acid sequence of SEQ ID NO: 1; or (c) is contained in plasmid pOPHNN10787 which is contained in E. coli DSM 17765.

12. The isolated polynucleotide of claim 10, having at least one mutation in the mature polypeptide coding sequence of SEQ ID NO: 1, in which the mutant nucleotide sequence encodes a polypeptide consisting of amino acids 36 to 334 of SEQ ID NO: 2.

13. A nucleic acid construct comprising the polynucleotide of claim 10, operably linked to one or more control sequences that direct the production of the polypeptide in an expression host.

14. A recombinant expression vector comprising the nucleic acid construct of claim 13.

15. A recombinant host cell comprising the nucleic acid construct of claim 13.

16. A method for producing the polypeptide of claim 1, comprising (a) cultivating a cell, which in its wild-type form is capable of producing the polypeptide, under conditions conducive for production of the polypeptide; and (b) recovering the polypeptide.

17. A method for producing the polypeptide of claim 1, comprising (a) cultivating a host cell comprising a nucleic acid construct comprising a nucleotide sequence encoding the polypeptide under conditions conducive for production of the polypeptide; and (b) recovering the polypeptide.

18. A method for producing a polynucleotide having a mutant nucleotide sequence, comprising (a) introducing at least one mutation into the mature polypeptide coding sequence of SEQ ID NO: 1, wherein the mutant nucleotide sequence encodes a polypeptide at least comprising amino acids 36 to 334 of SEQ ID NO: 2; and (b) recovering the polynucleotide comprising the mutant nucleotide sequence.

19. A method for producing the polypeptide of any of claim 1, comprising (a) cultivating a transgenic plant or a plant cell comprising a polynucleotide encoding a polypeptide having organophosphatase activity of the present invention under conditions conducive for production of the polypeptide; and (b) recovering the polypeptide.

20. A method for producing the polypeptide of claim 1, comprising (a) cultivating an algae comprising a polynucleotide encoding a polypeptide having organophosphatase activity of the present invention under conditions conducive for production of the polypeptide; and (b) recovering the polypeptide.

21. A use of the polypeptide according to claim 1, for hydrolysing an organophosphorus molecule.

22. A composition for hydrolysing an organophosphorus molecule, said composition comprising a polypeptide according to claim 1, and one or more acceptable carriers.

23. A composition for hydrolysing an organophosphorus molecule, said composition comprising a host cell according to claim 15.

24. A transgenic plant which produces a polypeptide according to claim 1.

25. A method for hydrolysing an organophosphorus molecule, said method comprising exposing the organophosphorus molecule to the polypeptide according to claim 1.


SEQUENCE LISTING (11 sequences)

SEQ ID NO: 1 - DNA, 1005 bp, Ralstonia pickettii
atgcgtttgcttacttctgtcgccgtgatagccgtggccgccgcgatggcgacgtctttcggagtcgcccaagcccaaactgcaacttcagccgctaccgctgccacgcaacaacgcacccaagtgcccggttactaccgtatggcgctgggtgaccacgtggtgaccgcgctctacgacggctacaccgatctgcccgccaagactctgcttggcctgaacgcgcaatcggtgcagagcctgctggcgcggatgtttgtatcgaacacgccgggcatgcagacggcggtgaacggctttctgatcgacacgggtgccaagcgcatcctcgttgatacggggtcgggcacgtgctttggcccgacgatgggcggcctggccggcaacgtccgcgcctccggctatcagcccgagcagatcgatgccgtgctgctgacgcacctgcatccggaccacgcatgcggcctcctgacgccgcaaggtcaaccggcctttccgaatgcgcaggtgtatgtcgccgcgccggaagccgatttctggctgagcgaaacgattgccgcttccaaaccgagcgacatgcaaccgttcttcaagatggcgcgcgactcagtggcgccatatgccgctgcgggcaagctcaagcaattcaagccgggtgacgaggtggtgcccggcgtacggtcggtgcgcgccaacgggcacacgccggggcatagcggttacctgttcgcatcgaagggacacagcctgctggtgtggggcgatatcgtgcacagccacgccgtgcagttccagcaccccgaggtcgcgttcgaatatgacgtcgacaagaaggctgctgtcgccacgcgccgcaagctgtttgcggaagcggcgcgagacaagctctgggtggccggcgctcatcttccgttcccggggctggggcacgtgcgccgcgagggcaacgccttcgcctgggtgccgatcgaatacggcccgctgcgcacagaccgcgctgattga

SEQ ID NO: 2 - protein, 334 aa, Ralstonia pickettii
MRLLTSVAVIAVAAAMATSFGVAQAQTATSAATAATQQRTQVPGYYRMALGDHVVTALYDGYTDLPAKTLLGLNAQSVQSLLARMFVSNTPGMQTAVNGFLIDTGAKRILVDTGSGTCFGPTMGGLAGNVRASGYQPEQIDAVLLTHLHPDHACGLLTPQGQPAFPNAQVYVAAPEADFWLSETIAASKPSDMQPFFKMARDSVAPYAAAGKLKQFKPGDEVVPGVRSVRANGHTPGHSGYLFASKGHSLLVWGDIVHSHAVQFQHPEVAFEYDVDKKAAVATRRKLFAEAARDKLWVAGAHLPFPGLGHVRREGNAFAWVPIEYGPLRTDRAD

SEQ ID NO: 3 - DNA, 33 bp, Artificial
  PCR primer
ggattaatgcgtttgcttacttctgtcgccgtg

SEQ ID NO: 4 - DNA, 30 bp, Artificial
  PCR primer
ccgctcgagatcagcgcggtctgtgcgcag

SEQ ID NO: 5 - DNA, 33 bp, Artificial
  PCR primer
gtaggatccaccatgcgtttgcttacttctgtc

SEQ ID NO: 6 - DNA, 29 bp, Artificial
  PCR primer
cttcttaagtcaatcagcgcggtctgtgc

SEQ ID NO: 7 - DNA, 94 bp, Artificial
  PCR primer
ggggatccaccatgagattatcgacttcgagtctcttcctttccgtgtctctgctggggaagctggccctcgggcaaactgcaacttcagccgc

SEQ ID NO: 8 - protein, 12 aa, Artificial
  alignment sequence 1
ACMSHTWGERNL

SEQ ID NO: 9 - protein, 14 aa, Artificial
  aligment sequence 2
HGWGEDANLAMNPS

SEQ ID NO: 10 - protein, 7 aa, Ralstonia pickettii
ATQQRTQ

SEQ ID NO: 11 - protein, 7 aa, Ralstonia pickettii
TQQRTQV